(19) United States
(12) Patent Application Publication
(10) Pub. No.: US 2010/0036137 A1
(43) Pub. Date: Feb. 11, 2010

(54) PROCESS FOR THE PREPARATION OF ALKYLENE CARBONATES

(76) Inventors: Chao-Jun Li, Brossard (CA); Nicolas Dara Eghbali, Bordeaux (FR)

Correspondence Address: FULBRIGHT & JAWORSKI L.L.P., 600 CONGRESS AVE., SUITE 2400, AUSTIN, TX, 78701, US

(21) Appl. No.: 12/442,270

(22) PCT Filed: Sep. 21, 2007
(86) PCT No.: PCT/CA07/01705

§ 371 Date: Oct. 19, 2009

Related U.S. Application Data
(60) Provisional application No. 60/826,496, filed on Sep. 21, 2006.
(60) Provisional application No. 60/872,404, filed on Dec. 19, 2006.

(51) Int. Cl. C07D 317/08

(52) U.S. Cl. 549/230

(57) ABSTRACT

An aqueous process for preparing alkylene carbonates from alkenes and carbon dioxide is described herein. The process comprises the reaction of alkenes with a bromine source, a base and carbon dioxide. The aqueous process can be rendered catalytic by using an oxidant capable of in situ conversion of bromide into bromine.

### CROSS REFERENCE TO RELATED APPLICATIONS

The present application claims the benefit of U.S. Provisional Applications No. 60/826,496 and 60/872,404 filed on Sep. 21, 2006 and Dec. 19, 2006 respectively, the entire contents of which are incorporated by reference.

### FIELD OF THE INVENTION

The present disclosure relates generally to a process for the preparation of alkylene carbonates. More specifically, but not exclusively, the present disclosure relates to a process for the preparation of alkylene carbonates from alkenes and carbon dioxide.

### BACKGROUND OF THE INVENTION

The excess emission of greenhouse gases (e.g. carbon dioxide) into the atmosphere has caused increasing health and environmental concerns. Although greenhouse gases are produced both naturally as well as from human activities, it is believed that the excess burning of fossil fuels, releasing carbon dioxide (CO₂) as an end product, is a predominant factor in tilting the natural balance. Presently, the CO₂ level in the atmosphere has reached 381 parts per million (ppm). Due to recent environmental considerations and the build-up of such green house gases in the atmosphere, the use of carbon dioxide as a raw material for organic synthesis has become of major interest.¹

A number of chemical processes have been developed to incorporate CO₂ in the synthesis of organic chemicals and materials.² In fact, a portion of the industrial production of urea, salicylic acid, carbonate and polycarbonate (traditionally produced using the well known “phosgene process”) is already based on the use of CO₂.³ Moreover, other processes using CO₂ as a raw material are currently under development, including the incorporation of CO₂ in the synthesis of lactones and carboxylic acids.⁴

A common approach to convert CO₂ into polycarbonates is based on the reaction of epoxides with CO₂ using a variety of catalysts. The coupling reaction of CO₂ with epoxides to generate cyclic carbonates and polycarbonates has witnessed important developments in recent years, largely due to the many existing and potential applications and properties of such carbonates and cyclic carbonates.^{5,6} Polycarbonates comprise versatile biodegradable polymers that can be readily produced from cyclic carbonates.⁶ The use of organic and inorganic reagents as Lewis bases, Lewis acids or bifunctional catalysts for the preparation of cyclic carbonates is referenced in several reviews.⁵ Although quite efficient, the coupling reaction usually requires the preliminary synthesis of an epoxide; an additional step that frequently calls-upon expensive and often toxic reagents, as well as a separate purification/separation procedure.⁷

A further approach to cyclic carbonates starting from olefins is disclosed in U.S. Pat. No. 3,025,305, issued to Verdol J. A. on Mar. 13, 1962. This approach, referred to as “oxidative carboxylation”, calls upon the direct oxidation of olefins in the liquid phase with gaseous mixtures of carbon dioxide and a molecular oxygen-containing gas.

Yet a further approach to cyclic carbonates starting from olefins is disclosed in U.S. Pat. Nos. 4,325,874 and 4,483,994 issued to Jacobsen S. E. on Apr. 20, 1982 and Nov. 20, 1984 respectively. The former patent teaches a process in which an olefin is reacted with carbon dioxide in the presence of iodine or an iodide compound and an oxide or a weak acid salt of thallium (Iii). The latter patent teaches a process in which an olefin is reacted with carbon dioxide, a thallic oxide and a weak acid, or, a weak acid thallic salt, in an aqueous organic solvent medium. Both processes comprise the in situ epoxidation of the olefin. Additional approaches to cyclic carbonates starting from olefins have been disclosed by Aresta⁸, Srivastava⁹ and Arai.¹⁰

Yet additional approaches to alkylene carbonates starting from olefins have been independently disclosed in U.S. Pat. No. 4,009,183 issued to Fumagalli et al. on Feb. 22, 1977; U.S. Pat. No. 4,224,223 issued to Wheaton et al. on Sep. 23, 1980; and U.S. Pat. No. 4,247,465 issued to Kao et al. on Jan. 27, 1981. These approaches comprise the reaction of an olefin with carbon dioxide in the presence of an oxidant and a suitable catalytic system. These approaches are based on the in situ formation of an iodohydrin.

Although significant efforts have been made to develop effective procedures for the preparation of cyclic carbonates, many of the prior art reported processes suffer from at least one of the following drawbacks: very high working temperatures and pressures; the use of toxic and expensive reagents which often give rise to the concomitant formation of undesired side-products; and the use of corrosive halogen reagents.

Environmental concerns, due to the extensive use of volatile organic solvents in many of the currently used processes, has led to an increased interest in, and need for the development of alternative or novel chemical processes, especially those that rely upon environmentally more friendly solvents such as water, supercritical CO₂, and ionic liquids.¹¹⁻¹⁴

The present invention refers to a number of documents, the content of which is herein incorporated by reference in their entirety.

### SUMMARY OF THE INVENTION

The present disclosure relates to an aqueous process for the preparation of alkylene carbonates from alkenes and carbon dioxide. The process proceeds smoothly and efficiently under mild reaction conditions. In an embodiment of the present disclosure, alkylene carbonates are obtained from alkenes and carbon dioxide by means of an aqueous process in which the use of toxic, expensive or corrosive reagents is at least substantially eliminated.

More specifically, as broadly claimed, the present disclosure relates to an aqueous process for the preparation of alkylene carbonates, the process comprising reacting an alkene with carbon dioxide in the presence of a suitable halogen source and a suitable amine base. In an embodiment of the present disclosure, the halogen source is a bromine source.

In an embodiment, the present disclosure relates to an aqueous catalytic process for the preparation of alkylene carbonates, the process comprising reacting an alkene with carbon dioxide in the presence of a suitable halogen source, a suitable amine base and an oxidant. In an embodiment of the present disclosure, the halogen source is a bromine source.

In an embodiment, the present disclosure relates to an aqueous catalytic process for the preparation of alkylene carbonates, the process comprising reacting an alkene with carbon dioxide in the presence of a suitable halogen source, a suitable amine base, an oxidant and optionally a metal or metal-based co-catalyst.

In an embodiment, the present disclosure relates to a substantially metal-free aqueous catalytic process for the preparation of alkylene carbonates, the process comprising reacting an alkene with carbon dioxide.

The foregoing and other objects, advantages and features of the present disclosure will become more apparent upon reading of the following non-restrictive description of illustrative embodiments thereof, given by way of example only.

### DETAILED DESCRIPTION OF ILLUSTRATIVE EMBODIMENTS

In order to provide a clear and consistent understanding of the terms used in the present specification, a number of definitions are provided below. Moreover, unless defined otherwise, all technical and scientific terms as used herein have the same meaning as commonly understood by one of ordinary skill in the art to which this invention pertains.

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “about” is used to indicate that a value includes an inherent variation of error for the device or the method being employed to determine the value.

The term “aqueous” is meant to include any type of reaction medium comprising water. This includes, but is not limited to, systems comprising water and optionally one or more co-solvents.

The present description refers to a number of chemical terms and abbreviations used by those skilled in the art. Nevertheless, definitions of selected terms are provided for clarity and consistency.

Abbreviations: DBU: 1,8-diazabicyclo[5.4.0]undec-7-ene; NBS: N-bromosuccinimide; TBAB: tetrabutylammonium bromide; DMAP: 4-dimethylaminopyridine; DIEA: diisopropylethylamine (Hünig's base); TEA: triethylamine; and DABCO: 1,4-diazabicyclo[2.2.2]octane.

As used in this specification, the term “alkyl” refers to a univalent group derived by conceptual removal of one hydrogen atom from a straight or branched-chain acyclic or cyclic saturated hydrocarbon. Examples of alkyl groups include, but are not limited to, C₁₋₁₀ alkyl groups. Examples of C₁₋₁₀ alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, nonyl and decyl.

As used in this specification, the term “alkylene” refers to a C₁₋₁₀ bivalent group derived from a straight or branched-chain acyclic, cyclic, saturated, or unsaturated hydrocarbon by conceptual removal of two hydrogen atoms from different carbon atoms (i.e., —CH₂CH₂—, —CH₂CH₂CH₂CH₂—, —CH₂C(CH₃)₂CH₂—, —PhCHCH₂—, etc.).

In an embodiment of the present disclosure, the alkyl and alkylene groups may be substituted by replacing one or more hydrogen atoms by alternative non-hydrogen groups. These include, but are not limited to, halo, hydroxy, alkyloxy, amino and SO₃H.

The present disclosure relates to an aqueous process for the preparation of alkylene carbonates from alkenes and carbon dioxide. More specifically, but not exclusively, the present disclosure relates to an aqueous catalytic process for the preparation of alkylene carbonates from alkenes and carbon dioxide. The process calls upon both the in situ formation of a halohydrin and the use of a base. In an embodiment, the present disclosure relates to a substantially metal-free aqueous catalytic process for the preparation of alkylene carbonates from alkenes and carbon dioxide.

The catalytic process of the present disclosure calls upon both the in situ formation of a halohydrin and the use of a base. A non-limiting example of such a base comprises DBU. In an embodiment of the present disclosure, the halohydrin comprises bromohydrin.

The stoichiometric process for the conversion of various terminal alkenes to the corresponding alkylene carbonates, using NBS as a source for bromine and DBU as the base, was carried out as generally illustrated hereinbelow in Equation 1.

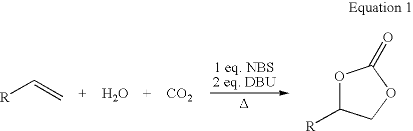

NBS was reacted with several terminal alkenes in an aqueous solution containing DBU. The results are summarized hereinbelow in Table 1 (entries 1-3). The reaction proceeded effectively using styrene as the terminal alkene, affording a mixture of styrene carbonate (85%) and bromohydrin (˜10%) after 2 hours at 60° C. No starting material could be detected at the end of the reaction due to the fast rate of bromohydrin formation. It is believed that the phenyl ring exerts an activating effect on the carbonation of styrene (Table 1, entry 1). An excess amount of base (2 eq. DBU) was used since the final cyclization step, terminating in carbonate formation, comprises both deprotonation of the alcohol functionality of the bromohydrin and the neutralization of the hydrobromic acid generated in situ. When applied to terminal alkenes such as 1-hexene or 1-octene, carbonate formation still proceeds smoothly even though reaction rates were observed to decrease (Table 1, entries 2 and 3).

TABLE 1
Conversion of terminal alkenes to alkylene carbonates.

| | | | | | | NMR |
|---|---|---|---|---|---|---|
| | | Bromide | Catalyst | DBU | Reaction | Yield |
| Entry | Alkene | Catalyst | (eq.) | (eq.) | Time (h) | (%)^{e} |
| 1^{a} | Styrene | NBS | 1 | 2 | 2 | 85 (98) |
| 2^{b} | 1-Hexene | NBS | 1 | 2 | 5 | 80 (98) |
| 3^{a} | 1-Octene | NBS | 1 | 2 | 5 | 65 (89) |
| 4^{c} | Styrene | NBS | 0.1 | 0.15 | 15 | 26 (36) |
| 5^{c} | Styrene | TBAB | 0.1 | 0.15 | 15 | 70 (88) |
| 6^{c,d} | 1-Hexene | TBAB | 0.1 | 0.15 | 15 | 31 (72) |
| 7^{c} | Styrene | NaBr | 0.1 | 0.15 | 15 | 63 (78) |

^{a}Reaction conditions: 1.5 mmol of alkene; 1.5 mmol of NBS; 3 mmol of DBU; 1 mL of H₂O; a CO₂ pressure of 250 psi, 60° C.
^{b}1 mmol of alkene was used and the temperature was kept at 42° C.
^{c}Reaction conditions: 1.5 mmol of alkene; 0.15 mmol of NBS, TBAB or NaBr; 0.2 mmol of DBU; 1 mL of H₂O₂ (30%); CO₂ pressure of 250 psi, 45-55° C.
^{d}The reaction temperature was set to 40° C.
^{e}Based on the alkene; The number in brackets refers to the conversion as calculated by ¹H NMR.

The results from the aqueous stoichiometric process for the conversion of various functionalized terminal alkenes to the corresponding alkylene carbonates, using NBS as a source for bromine and DBU as the base, are summarized hereinbelow in Table 2 (entries 1-5).

TABLE 2
Conversion of functionalized terminal alkenes to alkylene carbonates.

| Entry | Alkene (R) | Reaction Time (h) | NMR Yield (%)^{c} |
|---|---|---|---|
| 1^{a} | Ph | 3 | 89 (98) |
| 2^{a} | 4-MePh | 3 | 91 (98) |
| 3^{a} | 4-SO₃Ph | 6 | 95 (100) |
| 4^{a} | PhCH₂ | 4 | 61 (98) |
| 5^{a} | 3-MeOPhCH₂ | 4 | 45 (80) |
| 6^{b} | CH₃(CH₂)₃ | 5 | 85 (100) |
| 7^{b} | CH₃(CH₂)₅ | 5 | 63 (78) |

^{a}Reaction conditions: 1.5 mmol of alkene; 1.5 mmol of NBS; 3 mmol of DBU; 1 ml of H₂O; a CO₂ pressure of 250 psi, 60° C.
^{b}1 mmol of alkene was used and the temperature was kept at 42° C.
^{c}Based on the alkene; The number in brackets refers to the conversion as calculated by ¹H NMR.

A catalytic process in which the generated hydrobromic acid is converted in situ into a source of bromine was subsequently developed. In an embodiment of the present disclosure, a metal-free aqueous catalytic process was developed (Equation 2).

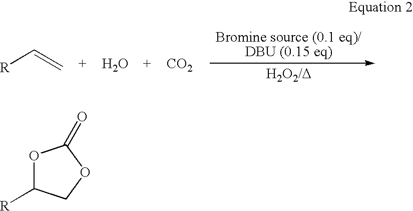

As illustrated hereinbelow in Scheme 1, instead of being neutralized, the generated hydrobromic acid is oxidized in situ to bromine and/or hypobromous acid, a reagent known to react with alkenes under aqueous conditions to provide the corresponding bromohydrin. The process calls upon the use of a catalytic amount of a bromine source to initiate the carbonation process. In an embodiment of the present disclosure, the bromine source is selected from the group consisting of NBS, TBAB and NaBr. It is believed to be within the capacity of a skilled technician to select other suitable bromine sources. In an embodiment of the present disclosure, inexpensive oxidants such as hydrogen peroxide, persulfate or molecular oxygen were examined. It is believed to be within the capacity of a skilled technician to select other suitable oxidants or oxidation systems.

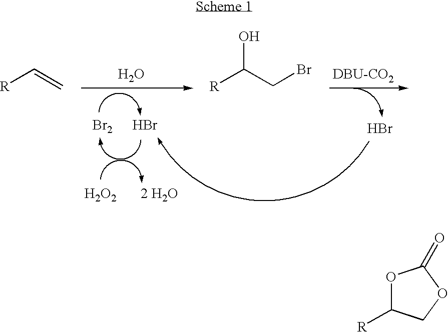

Following the formation of the bromohydrin intermediate and its subsequent reaction with carbon dioxide, the liberated bromide ions are converted (i.e. oxidized) into bromine. In an embodiment of the present disclosure, the bromide ions are converted into bromine by reaction with hydrogen peroxide. In the process, the hydrogen peroxide reagent is converted (i.e. reduced) into water.

The conversion of bromide ions into bromine by means of reaction with hydrogen peroxide is illustrated hereinbelow and proceeds via the reduction of the hydrogen peroxide to water (Equation 3).

Br⁻+H₂O₂→BrOH+HO⁻

BrOH+Br⁻→Br₂+HO⁻ Equation 3

In an embodiment, the present disclosure relates to a catalytic process that uses NBS (0.1 eq.) as the bromine source, DBU (0.15 eq.) as the base and excess amounts (5 equivalents) of an aqueous hydrogen peroxide solution (30%) for the conversion of bromide (i.e. hydrogen bromide) into bromine (Table 1, entry 4). Even though some of the desired carbonation product was obtained, the observed yield was low, most likely due to the high reactivity of NBS toward H₂O₂.

In a further embodiment, the present disclosure relates to a catalytic process that uses TBAB (0.1 eq.) as the bromine source, DBU (0.15 eq.) as the base and excess amounts (5 equivalents) of an aqueous hydrogen peroxide solution (30%) for the conversion of bromide (i.e. hydrogen bromide) into bromine (Table 1, entries 5 and 6).

In yet a further embodiment, the present disclosure relates to a catalytic process that uses NaBr (0.1 eq.) as the bromine source and DBU (0.15 eq.) as the base and excess amounts (5 equivalents) of an aqueous hydrogen peroxide solution (30%) for the conversion of bromide (i.e. hydrogen bromide) into bromine (Table 1, entry 7).

The catalytic processes comprising the use of either TBAB or NaBr as the bromine source proved to be effective for converting styrene into the corresponding styrene carbonate (70% and 63% respectively). Small amounts of starting material (styrene, 12%) and styrene bromohydrin (8%) were also observed.

The catalytic process for the conversion of various functionalized terminal alkenes to the corresponding alkylene carbonates in an aqueous solution comprising DBU, using NBS, NaBr or TBAB as a source for bromine and using excess amounts (5 equivalents) of an aqueous hydrogen peroxide solution (30%) for the conversion of bromide (i.e. hydrogen bromide) into bromine was also carried out (Table 3, entries 4 and 5).

TABLE 3
Catalytic process for the conversion of terminal and functionalized
terminal alkenes to alkylene carbonates.

| | | Bromide | Reaction Time | NMR Yield |
|---|---|---|---|---|
| Entry | Alkene (R) | Catalyst | (h) | (%)^{c} |
| 1^{a} | Ph | NBS | 15 | 26 (36) |
| 2^{a} | Ph | NaBr | 15 | 65 (80) |
| 3^{a} | Ph | TBAB | 15 | 70 (89) |
| 4^{a} | 4-MePh | TBAB | 15 | 72 (90) |
| 5^{a} | 4-SO₃Ph | TBAB | 15 | 89 (98) |
| 6^{b} | CH₃(CH₂)₃ | TBAB | 20 | 47 (72) |
| 7^{b} | CH₃(CH₂)₅ | TBAB | 20 | 27 (78) |

^{a}Reaction conditions: 1.5 mmol of alkene; 0.15 mmol of NBS, NaBr or TBAB; 0.2 mmol of DBU; 1 mL of H₂O₂ (30%); CO₂ pressure of 250 psi, 45-55° C.
^{b}1 mmol of alkene was used and the temperature was kept at 40° C.
^{c}Based on the alkene; The number in brackets refers to the conversion as calculated by ¹H NMR.

The catalytic process of the present disclosure comprises both the in situ formation of a stable halohydrin as well as the use of a base. In an embodiment of the present disclosure, the base comprises an organic base. In an embodiment of the present disclosure, the organic base comprises an amine base. In an embodiment of the present disclosure, the amine base comprises DBU. Various amine bases were tested as potential carbon dioxide activators for the preparation of alkylene carbonates from alkenes and carbon dioxide (Table 4). Non-limiting examples of amine bases as contemplated by the present disclosure include DBU, DMAP, DIEA, TEA, DABCO, 1-methylimidazole, pyridine, N,N,N′,N″,N″-pentamethyldiethylenetriamine, N-methyldiphenylamine, N,N-dimethylaniline, and N,N,N′,N′-tetramethyldiaminomethane. It is believed to be within the capacity of a skilled technician to select other suitable amine bases.

TABLE 4
Effect of organic amine base on the catalytic process for
the conversion of terminal alkenes to alkylene carbonates.

| | | NMR Yield |
|---|---|---|
| Entry^{a} | Organic Amine Base | (%)^{b} |
| 1 | DBU | 17 (20) |
| 2 | DMAP | 9 (10) |
| 3 | DIEA | 7.5 (10) |
| 4 | TEA | >1 (5) |
| 5 | 1-Methylimidazole | >1 (5) |
| 6 | Pyridine | NR |
| 7 | N,N,N′,N″,N″-pentamethyldiethylenetriamine | NR |
| 8 | N-methyldiphenylamine | NR |
| 9 | DABCO | NR |
| 10 | N,N-dimethylaniline | NR |
| 11 | N,N,N′,N′-tetramethyldiaminomethane | NR |

^{a}Reaction conditions: 3 mmol of styrene (0.2 mL, 1 eq.); 9 mmol of H₂O₂ (1 mL, 3 eq.); 0.7 mmol of TBAB (250 mg, 0.25 eq.); 1 mmol of organic amine base (0.3 eq.); CO₂ pressure of 250 psi; a reaction time of 2 h (the reaction time was set at 2 hours for all bases tested for screening purposes; the reactions were not carried out to completion); and a reaction temperature of 42° C.
^{b}Based on the alkene; The number in brackets refers to the conversion as calculated by ¹H NMR.

The catalytic processes comprising the use of either DBU, DMAP or DIEA as the amine base (Table 4, entries 1, 2 and 3) proved to be effective for converting styrene into the corresponding styrene carbonate, with DBU being the most effective base.

Aqueous hydrogen peroxide (30%) comprises an effective oxidant for the conversion of bromide into bromine. However, in view of the apparent reactivity of NBS toward H₂O₂, other oxidants were examined. Styrene was converted into the corresponding styrene carbonate by means a catalytic process comprising the use of excess amounts of an aqueous sodium persulfate solution for the conversion of bromide (i.e. hydrogen bromide) into bromine, using either TBAB or NBS as the bromine source and using an aqueous solution of DBU as the base. The catalytic process afforded styrene carbonate in modest yields (˜14%) together with large amounts of unreacted starting material. Sodium persulfate appears to be a less effective oxidizing agent for the conversion of bromide into bromine.

Aqueous hydrogen peroxide and sodium persulfate solutions comprise attractive oxidants for the catalytic preparation of alkylene carbonates from alkenes and carbon dioxide. Other suitable oxidants are known in the art and are within the capacity of a skilled technician. Non-limiting examples of such other oxidants include oxygen gas and the NO/NO₂ redox couple. It is believed to be within the capacity of a skilled technician to select other suitable oxidants.

The present invention is illustrated in further detail by the following non-limiting examples.

### EXPERIMENTAL

General. Reagents were obtained commercially from Aldrich Chemical Co. and were used without further purification unless otherwise noted. ¹H NMR and ¹³C NMR spectra were acquired using Varian 300 MHz and 75 MHz spectrometers respectively, and referenced to the internal solvent signals. Unless otherwise noted, proton chemical shifts were internally referenced to the residual proton resonance in CDCl₃ (δ 7.26 ppm). Unless otherwise noted, carbon chemical shifts were internally referenced to the deuterated solvent signals in CDCl₃ (δ 77.2 ppm). Silica gel (60 Å, 230-400 mesh) used in flash column chromatography was obtained from Silicycle and was typically used as received. A typical eluant system was comprised of an ethyl acetate/hexane mixture. References following compound names indicate literature articles where ¹H and ¹³C NMR data have previously been reported.

In an embodiment of the present disclosure, the carbonation reactions were carried out in an autoclave (Parr reactor) pressurized with CO₂ at an overall pressure of 250 psi. The carbonation reactions can be carried out at various CO₂ pressures. The determination of other suitable CO₂ pressures is within the capacity of a skilled technician.

### Example 1

### Typical Procedure for the Stoichiometric Conversion of Alkenes to Alkylene Carbonates using NBS and DBU

NBS (266 mg, 1.5 mmol) was added to a vial comprising a magnetic stirrer, and partially dissolved using water (1 mL). DBU (0.35 mL, 2.4 mmol) was added to the mixture using a syringe, followed by the immediate addition of the alkene (1.5 mmol). The reaction mixture was not stirred until the onset of the reaction. The vial was then placed in a stainless steal autoclave (Parr reactor) which was pressurized with CO₂ at an overall pressure of 250 psi. The reactor was not purged prior to pressurization. The reaction temperature was maintained at about 40 to about 60° C. (depending on the nature of the alkene) using a Parr temperature controller. After 2-6 hours of reaction time (depending on the nature of the alkene), the reactor was cooled to room temperature and depressurized. Ethyl acetate (1 mL) was used to extract the organic material. Following purification by flash chromatography, the product was characterized by ¹H and ¹³C NMR.

Styrene carbonate (15): Isolated yield 85%; ¹H NMR (CDCl₃, 300 MHz, ppm): δ 4.26 (t, 1H), 4.71 (t, 1H), 5.59 (t, 1H), 7.34 (m, 5H); ¹³C NMR (CDCl₃, 75 MHz, ppm): δ 71.1, 77.9, 125.8, 129.1, 129.6, 135.7, 154.8.

1-Hexene carbonate (15): Isolated yield 80%; ¹H NMR (CDCl₃, 300 MHz, ppm): δ 0.86 (t, 2H), 1.29 (m, 4H), 1.32 (m, 2H), 4.01 (t, 1H), 4.47 (t, 1H), 4.62 (m, 1H); ¹³C NMR (CDCl₃, 75 MHz, ppm): δ 13.7, 22.15, 26.3, 33.4, 69.3, 77.0, 155.0.

1-Octene carbonate: Isolated yield 65%; ¹H NMR (CDCl₃, 300 MHz, ppm): δ 0.88 (t, 3H, J=6.5 Hz), 1.26 (m, 8H), 1.72 (m, 2H), 4.01 (t, 1H, J=6.9), 4.50 (t, 1H, J=7.2), 4.69 (m, 1H); ¹³C NMR (CDCl₃, 75 MHz, ppm): δ 14.0, 22.50, 24.32, 28.80, 31.50, 38.90, 69.40, 77.03, 155.10.

4-Methyl styrene carbonate (16): Isolated yield 91%; ¹H NMR (CDCl₃, 300 MHz, ppm): δ 2.36 (s, 3H), 4.32 (t, 1H, J=8.1 Hz), 4.75 (t, 1H, J=8.4 Hz), 5.62 (t, 1H, J=7.95 Hz), 7.23 (s, 4H). ¹³C NMR (CDCl₃, 75 MHz, ppm): δ 29.4, 71.4, 78.3, 126.2, 130.1, 132.9, 140.1, 155.1. Purification was achieved by preparative TLC using toluene as the eluant system.

Allyl benzene carbonate: Isolated yield 61%; ¹H NMR (CDCl₃, 300 MHz, ppm): δ 3.07 (dd, 2H, J=14.1, 13.8 Hz), 4.168, (t, 1H, J=6.8 Hz), 4.44 (t, 1H, J=8.1 Hz), 4.93 (m, 1H), 7.30 (m, 5H). ¹³C NMR (CDCl₃, 75 MHz, ppm): δ 39.8, 68.7, 77.0, 127.8, 129.2, 129.5, 134.1, 154.9. Purification was achieved using an ethyl acetate/hexane (1:5) eluent system followed by a THF or an ethyl acetate/hexane (1:1) eluent system.

Allyl anisole carbonate: Isolated yield 45%; ¹H NMR (CDCl₃, 300 MHz, ppm): δ 3.8 (s, 3H), 3.94 (m, 2H), 4.33 (t, 1H, J=8.4 Hz), 4.73 (t, 1H, J=8.4 Hz), 5.6 (t, 1H, J=8.1 Hz), 7.11 (s, 4H). ¹³C NMR (CDCl₃, 75 MHz, ppm): δ 27.9, 55.4, 71.1, 78.1, 114.6, 127.8, 129.5, 154.9, 160.7. LRMS [(M+Na)⁺] C₁₁H₁₂O₄Na: 231.0.

4-Styrene carbonate sulfonic acid sodium salt: Isolated yield 89%; ¹H NMR (D₂O, 400 MHz, ppm): δ 4.39 (t, 1H, J=8.4 Hz), 4.86 (t, 1H, J=8.6 Hz), 5.86 (t, 1H, J=8.2 Hz), 7.61 (q, 4H, J=8 Hz). ¹³C NMR (D₂O, 100 MHz, ppm): δ 71.6, 78.2, 126.2, 127.0, 138.8, 144.3, 156.9. HRMS (FTMS) C₉H₇O₆S calculated: 242.99633; found: 242.99659. The use of either KHCO₃ or NaHCO₃ proved to be efficient alternatives to the use of DBU.

### Example 2

### Typical Procedure for the Catalytic Conversion of Alkenes to Alkylene Carbonates Using Aqueous Hydrogen Peroxide (30%)

TBAB (50 mg, 0.15 mmol) was added to a vial comprising a magnetic stirrer, and dissolved using a 30% H₂O₂ solution (1 mL, 9 mmol, 6 eq.). DBU (0.03 mL, 0.2 mmol) was added to the mixture using a syringe, followed by the immediate addition of the alkene (1.5 mmol). The vial was then placed in a stainless steal autoclave (Parr reactor) which was pressurized with CO₂ at an overall pressure of 250 psi. The reactor was not purged prior to pressurization. The reaction temperature was maintained at about 40° C. to about 60° C. (depending on the nature of the alkene) using a Parr temperature controller. After an average of about 15 hours of reaction time, the reactor was cooled to room temperature and depressurized. Ethyl acetate (0.3 mL) was used to extract the organic material. The crude product was analyzed by ¹H NMR.

### Example 3

### Typical Procedure for the Conversion of Ethylene to Ethylene Carbonate Using NBS and KHCO₃

KHCO₃ (500 mg, 5 mmol, 2 eq.) was dissolved in water (3 mL) and NBS (2.5 mmol, 1 eq.) was added to the solution. The vial was placed in a reactor which was subsequently sealed. After purging the reactor, the vacuum was replaced with ethylene gas (40 psi). The reactor was then pressurized with CO₂ gas (overall pressure of 500 psi) and heated to 60° C. for a period of 4 h. Following the reaction, the reactor was cooled to 10° C. using ice and depressurized. The reaction mixture was extracted using chloroform and subsequently analyzed by ¹H NMR. Ethylene carbonate: Yield 29% (based on the quantity of NBS); ¹H NMR (CDCl₃, 300 MHz, ppm): δ 4.51 (s, 4H). ¹³C NMR ((CDCl₃, 75 MHz, ppm): δ 64.7. GCMS C₃H₄O₃: found 88.1.

It is to be understood that the disclosure is not limited in its application to the details of construction and parts as described hereinabove. The disclosure is capable of other embodiments and of being practiced in various ways. It is also understood that the phraseology or terminology used herein is for the purpose of description and not limitation. Hence, although the present disclosure has been described hereinabove by way of illustrative embodiments thereof, it can be modified without departing from the spirit, scope and nature as defined in the appended claims.

### REFERENCES

1. Anastas, P. T.; Warner, J. C. Green Chemistry: Theory and Practice, Oxford University Press, 1998. Omae, I. Catal. Today, 2006, 115, 33. Zevenhoven, R. Eloneva, S. Teir, S. Catal. Today 2006, 115, 73.
2. Behr, A. Chem. Ing. Tech. 1985, 10, 16.
3. Shaikh, A. G. and Sivaram, S. Chem Rev. 1996, 96, 951-976. Marks, T. J. et al. Chem Rev. 2001, 101, 953-996. Sugimoto, H; Inoue, S. J. Pol. Sci.: Part A: pol. Chem. 2004, 42, 5561. Behr, A. Chem.-Ing.-Tech. 1985, 57, 893-903.
4. Behr, A. Angew. Chem. 1988, 100, 681-698.
5. Darensbourg, D. J. and Holtcamp, M. W. Coordination Chemistry Reviews, 1996, 153, 155-174. Yoshida, M.; Ihara, M. Chem. Eur. J. 2004, 10, 2886.
6. Clements, J. H. Int. Eng. Chem. Res. 2003, 42, 663.
7. As an example, the synthesis of tigliane-daphnane analogs reported by Wender was based on an epoxidation and carbocyclization sequence. Wender, P. A. and McDonald, F. E. Tetrahedron Lett. 1990 31, 3691.
8. Aresta, M. and Quaranta, E. J. Mol. Catal. A: Chem. 1987, 41, 355. Aresta, M.; Dibenedetto, A. and I. Tommasi, Appl. Organomet. Chem. 2000, 14, 799. Aresta, M. and Dibenedetto, A. J. Mol. Catal. A: Chem., 2002, 182-183, 399-409.
9. Srivastava, R.; Srinivas, D. and Ratnasamy, P. Catal. Lett. 2003, 91, 133.
10. Sun, J.; Fujika, S. I.; Bhanage, B. M. and Arai, M. Catalysis communications 2004, 5, 83-87. Sun, J.; Fujika, S. I.; Bhanage, B. M. and Arai, M. Catalysis Today 2004, 93-95, 383-388. Sun, J.; Fujika, S. I.; Zhao, F.; Hasegawa, M. and Arai, M. Journal of Catalysis 2005, 230, 398-405. Sun, J.; Fujika, S. I. and Arai, M. J. of Organomet. chem. 2005, 690, 3490-3497.
11. Adam, D. Nature, 2000, 407, 938. Sheldon, R. Green Chem. 2005, 7, 267.
12. For a recent reviews, Li, C. J. Chem. Rev. 2005, 105, 3095. Lindstrom, U. M. Chem. Rev. 2002, 102, 2751. Clean Solvents: Alternative Media for Chemical Reactions and Processing: Chan, T. H.; Li, L.; Yang, Y.; Lu, W. ACS Symposium Series 819, 2002, 166.
Sinou, D. Adv. Synth. Catal. 2002, 334, 221. Yorimitsu, H.; Shinokubo, H. Oshima, K. Synlett 2002, 5, 674.
13. Jessop, P. G., Leitner, W., Eds. Chemical Synthesis Using Supercritical Fluids; Wiley-VCH: Weinheim-New York, 1999. Leitner, W. Acc. Chem. Res. 2002, 35, 746. Jiang, H. F. Curr. Org. Chem. 2005, 9, 289. Campestrini, S.; Tonellato, U. Curr. Org. Chem. 2005, 9, 31.
14. Representative literature reviews: Ionic liquids in Synthesis (Eds.: Wasserscheid, P.; Welton, T.), Wiley-VCH, Weinheim, 2002. Welton, T. Chem. Rev. 1999, 99, 2071-2083. Earle, M. J.; Seddon, K. R. Pure Appl. Chem. 2000, 72, 1391. Welton, T. Coord. Chem. Rev. 2004, 248, 2459. Welton, T.; Smith, P. J. Adv. Organomet. Chem. 2004, 51, 251. Sheldon, R. A.; Lau, R. M.; Sorgedrager, M. J.; Rantwijk, F. V.; Seddon, K. R. Green Chem. 2002, 4, 147. Muzart, J. Adv. Synth. Catal. 2006, 348, 275. Pandey, S. Anal. Chim. Acta 2006, 556, 38. Zhao, H.; Xia, S.; Ma, P. J. Chem. Technol. Biotechnol. 2005, 80, 1089.
15. For a recent characterization, see Jiang, J.-L.; Feixue, G.; Hua, R. and Qiu, X. J. Org. Chem. 2005, 70, 381-388.
16. Barlow, T.; Dipple, A. Chem. Res. Toxicol. 1998, 11, 44-53.

What is claimed is:

1. An aqueous process for the preparation of alkylene carbonates comprising:
reacting an alkene with a bromine source, a base and carbon dioxide.

2. The process of claim 1, further comprising separating the alkylene carbonate from the aqueous medium.

3. The process of claim 1, wherein the reaction is carried out at temperatures ranging from about 20° C. to about 90° C.

4. The process of claim 3, wherein the reaction is carried out at temperatures ranging from about 40° C. to about 60° C.

5. The process of claim 1, wherein the reaction is carried out at carbon dioxide partial pressures of at least one atmosphere.

6. The process of claim 5, wherein the reaction is carried out at carbon dioxide partial pressures ranging from about 15 psi to about 1000 psi.

7. The process of claim 6, wherein the reaction is carried out at carbon dioxide partial pressures ranging from about 200 psi to about 500 psi.

8. The process of claim 1, wherein the alkylene carbonate comprises the structure: 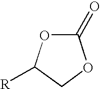
wherein R is selected from the group consisting of alkyl, substituted alkyl, phenyl and substituted phenyl.

9. The process of claim 1, wherein the bromine source is selected from the group consisting of NBS, TBAB, KBr and NaBr.

10. The process of claim 9, wherein the bromine source is TBAB.

11. The process of claim 1, wherein the base comprises an amine base.

12. The process of claim 11, wherein the amine base is selected from the group consisting of DBU, DMAP, DIEA, TEA, DABCO, 1-methylimidazole, pyridine, N,N,N′,N″,N″-pentamethyldiethylenetriamine, N-methyldiphenylamine, N,N-dimethylaniline, and N,N,N′,N′-tetramethyldiaminomethane.

13. The process of claim 12, wherein the amine is DBU.

14. An aqueous catalytic process for the preparation of alkylene carbonates comprising:
reacting an alkene with a bromine source, a base, carbon dioxide and an oxidant.

15. The process of claim 14, further comprising separating the alkylene carbonate from the aqueous medium.

16. The process of claim 14, wherein the reaction is carried out at temperatures ranging from about 20° C. to about 90° C.

17. The process of claim 16, wherein the reaction is carried out at temperatures ranging from about 40° C. to about 60° C.

18. The process of claim 14, wherein the reaction is carried out at carbon dioxide partial pressures of at least one atmosphere.

19. The process of claim 18, wherein the reaction is carried out at carbon dioxide partial pressures ranging from about 15 psi to about 1000 psi.

20. The process of claim 19, wherein the reaction is carried out at carbon dioxide partial pressures ranging from about 200 psi to about 500 psi.

21. The process of claim 14, wherein the oxidant is capable of converting bromide into bromine.

22. The process of claim 21, wherein the oxidant is a peroxide.

23. The process of claim 22, wherein the peroxide is hydrogen peroxide.

24. The process of claim 14, wherein the alkylene carbonate comprises the structure: 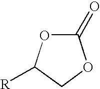
wherein R is selected from the group consisting of alkyl, substituted alkyl, phenyl and substituted phenyl.

25. The process of claim 14, wherein the bromine source is selected from the group consisting of NBS, TBAB, KBr and NaBr.

26. The process of claim 25, wherein the bromine source is TBAB.

27. The process of claim 14, wherein the base comprises an amine base.

28. The process of claim 27, wherein the amine base is selected from the group consisting of DBU, DMAP, DIEA, TEA, DABCO, 1-methylimidazole, pyridine, N,N,N′,N″,N″-pentamethyldiethylenetriamine, N-methyldiphenylamine, N,N-dimethylaniline, and N,N,N′,N′-tetramethyldiaminomethane.

29. The process of claim 28, wherein the amine is DBU.
